(19) United States
(12) Patent Application Publication
(10) Pub. No.: US 2024/0253065 A1
(43) Pub. Date: Aug. 1, 2024

(54) CENTRIFUGE FOR ROTATING A SAMPLE CARRIER

(71) Applicant: CYTENA GMBH, Freiburg (DE)

(72) Inventors: Julian RIBA, Freiburg (DE); David RAUCH, Freiburg (DE); Roman OPGENORTH, Freiburg (DE)

(21) Appl. No.: 18/565,289

(22) PCT Filed: May 30, 2022
(86) PCT No.: PCT/EP2022/064564

§ 371 Date: Nov. 29, 2023

(30) Foreign Application Priority Data
May 31, 2021 (LU) LU102813

(51) Int. Cl. B04B 15/06; A61L 2/00; A61L 2/26; B04B 5/04; B04B 7/02; B04B 7/08; B04B 15/02

(52) U.S. Cl. CPC B04B 15/06; A61L 2/0047; A61L 2/26; B04B 5/0407; B04B 7/02; B04B 7/08; B04B 15/02; A61L 2202/11

(57) ABSTRACT

The invention relates to a centrifuge for rotating a sample carrier, which has at least one container for receiving a liquid sample, with a rotatable rotor, which has at least one receiving section for receiving the sample carrier, and a rotor space in which the rotor is arranged. The centrifuge is characterized in that the centrifuge has at least one radiation source for emitting radiation into the rotor space with a wavelength of a maximum of 350 nm.

The invention relates to a centrifuge for rotating a sample carrier. In addition, the invention relates to a method for operating a centrifuge.

Centrifuges into which sample carriers can be introduced are known from the prior art. The sample carriers have at least one container that is used to receive a liquid sample. The centrifuge has a rotor with a receiving section that receives the sample carrier. The rotor is arranged in a rotor space of the centrifuge and rotates in this. As a result of the rotation of the rotor, part of the liquid sample is ejected from the sample holder due to the centrifugal force acting on the liquid sample. A desired biological particle thus remains in the container, which can then be further processed; in particular, liquid can be introduced into the container by a dispensing device, which can then be ejected again from the container. A rotor housing enclosing the rotor space has an outlet through which the ejected liquid and/or particles are removed from the rotor space. The centrifuge can be used, for example, to remove liquid and/or biological particles from the container, so that only desired cells and/or magnetic beads to which DNA or proteins are attached remain in the container. Such a centrifuge is known, for example, from WO 2015 0188 78 A1.

However, it may happen that at least part of the ejected sample cannot be completely removed from the rotor space. Since the part of the liquid sample that is not removed from the centrifuge can contain microorganisms or DNA, for example, there is a risk that liquid samples that are subsequently washed will be contaminated by the part of the liquid sample in the rotor space that has not been removed.

The object of the invention is therefore to provide a centrifuge in which the risk of contamination is low.

The object is achieved by a centrifuge for rotating a sample carrier which has at least one container for receiving a liquid sample, with a rotatable rotor which has at least one receiving section for receiving the sample carrier, and a rotor space in which the rotor is arranged, characterized in that the centrifuge has at least one radiation source for emitting radiation into the rotor space with a wavelength of a maximum of 350 nm (nanometers).

A further object of the invention is to provide a method with which the risk of contamination is minimized.

The object is achieved by a method when operating a centrifuge, wherein, in particular in a washing operation, a sample carrier which has at least one container for receiving a liquid sample is rotated in a rotor space by means of a rotor, characterized in that, in particular in a decontamination operation, radiation with a wavelength of a maximum of 350 nm (nanometers) is emitted into the rotor space.

The advantage of the invention is that it has been recognized that the risk of contamination is reduced if the rotor space is irradiated with radiation with a wavelength of a maximum of 350 nm. Such radiation has a sufficiently high level of energy to kill the biological particles contained in the liquid sample which were ejected from the sample carrier during the washing process and not removed from the rotor space.

Biological particles, in particular microorganisms, can particularly preferably be killed or inactivated if the wavelength is a maximum of 315 nm, preferably 280 nm. A lower limit of the radiation range can be at least 100 nm, in particular 200 nm. In such a case, the emitted radiation may have a wavelength in a range of 100 nm to 350 nm. It is particularly advantageous if the radiation emitted has a wavelength of 254 nm or is in a range between 260 nm and 265 nm.

The sample carrier has one or more containers and is used to receive a liquid sample. In particular, the liquid sample can be arranged in the container or containers. The sample carrier can be a microtiter plate. Microtiter plates can be designed with a different number of containers. Microtiter plates with 6 to 3456 containers are known, wherein microtiter plates with 96, 384 or 1536 containers are usually used. The sample carrier can have a receiving device into which the microtiter plate is inserted. The receiving section of the rotor can be designed such that a normal to a container opening is perpendicular to a rotor shaft.

This means that a longitudinal axis of the container of the sample carrier extends in a direction that is perpendicular to an extension direction of the axis of rotation of the rotor shaft. As a result, a plane having a surface of the receiving section on which the sample carrier is placed runs parallel to the axis of rotation of the rotor.

The liquid sample may include a liquid and biological particles. The biological particles may be microorganisms such as bacteria, archaea, yeasts, fungi, and viruses or cells, DNA, RNA, or proteins. The liquid sample may contain one or more of the aforementioned biological particles.

A radiation source means any device by means of which radiation with a wavelength of a maximum of 350 nm can be generated. This also includes devices by means of which radiation can be generated with wavelengths that only cover a portion of the aforementioned range. This means that the radiation source does not have to cover the entire radiation range, namely 100 nm to 350 nm.

The centrifuge can be operated in a washing operation. A washing operation is understood to be an operation of the centrifuge in which the rotor is rotated at a speed that is sufficiently high that, in particular, a portion of the liquid sample is ejected from the container or containers as a result of the acting centrifugal forces. However, it can be ensured that certain biological particles are retained in the container against the centrifugal force. After the washing operation has been completed, the desired biological particle thus remains in the container.

The biological particles can be retained, for example, via cell adhesion, magnetic forces, covalent chemical bonds, or similar. A washing operation is possible in which the bottom of the container is provided with a specific coating to which the biological particles adhere. In addition, a washing operation using magnetic beads is possible. In this washing operation, biomolecules such as DNA, RNA, or proteins are first bound to magnetic beads and then held on the bottom of the container using a magnet that is positioned below the container in the centrifuge. It is also possible for cells to be bound to magnetic beads.

In addition, a washing operation is known in which proteins are bound to the bottom of the container, which has previously been provided with a specific reagent. In another washing operation, cells, especially suspension cells, are first centrifuged vigorously so that a so-called pellet is formed at the bottom. The pellet then remains in the container when part of the liquid sample is ejected.

A decontamination operation is understood to be an operation in which radiation is emitted in order to kill or decompose the biological particles located in the rotor space.

The decontamination operation takes advantage of the fact that irradiating DNA and RNA with radiation of a certain wavelength ensures that they cannot be replicated further or decay over time. When cells are exposed to radiation of a certain wavelength, a structural change in the DNA is often observed, which is due to the formation of thymidine dimers. Two thymidines adjacent to one DNA strand are covalently linked via a cyclobutane ring. The mechanism is photoinduced cycloaddition. By absorbing a quantum of light, a thymidine molecule is excited and can thereby now interact with a thymidine in its basic state. The double bond between the C5 and C6 atoms of thymine is broken in both molecules. At the same time, single bonds form between the C5 atoms and the C6 atoms of both molecules. The consequence of such thymidine dimer formation is a change in the spatial shape of the DNA. Proteins that interact with DNA cannot pass through the resulting dimer. Therefore, transcription and replication come to a standstill at these sites.

The result is a centrifuge that can be operated either in a washing operation or a decontamination operation. In addition, a method for operating a centrifuge is provided.

In a particular embodiment, the rotor space can be delimited by a rotor housing. The rotor housing can have a shell. The shell can delimit the rotor space in the radial and/or tangential direction. The shell can be designed in several parts. The shell can have an upper shell and a lower shell, which can be releasably connected to one another. The upper shell is arranged on the lower shell. The upper and lower shells can be designed such that they delimit the rotor space, in particular in the radial, axial, and tangential directions. As a result, a compact rotor housing can be provided.

A particularly compact rotor housing is realized if the rotor housing, in particular the upper shell, has a cylindrical inner housing surface. The rotor housing can have the cylindrical inner housing surface in a normal plane that is normal to a rotor shaft described in more detail below and includes a part of the upper and/or lower shell. The inner housing surface is understood to be the surface of the rotor housing that directly delimits the rotor space. The cylindrical inner housing surface can be realized by appropriately designing the upper shell and/or lower shell. The cylindrical inner housing surface enables the rotor space in which the rotor rotates to be designed to be as compact as possible, i.e. the distance between the rotor and the rotor housing, in particular in the radial direction, is small.

The “radial”, “tangential” and “axial” direction is understood to mean a direction that refers to a central axis of the rotor housing. The central axis can run coaxially or parallel to a longitudinal axis of the centrifuge and/or a central axis of the rotor shaft. The central axis of the rotor shaft corresponds to the axis of rotation of the rotor shaft and thus of the rotor.

The rotor housing can be designed such that it prevents the radiation from escaping from the rotor space. To prevent radiation from escaping, the rotor housing can have a front wall and a rear wall. The front and rear walls delimit the rotor space, especially in the axial direction. The rotor space is thus delimited by the upper shell, the lower shell, and the front and rear walls. The front wall corresponds to the wall that is closer to a centrifuge holder than the rear wall. The previously described design of the rotor housing can easily prevent the radiation from escaping from the rotor space and hitting liquid samples that are arranged outside the rotor space. It also prevents, for example, a user of the centrifuge from coming into contact with the high-energy radiation.

In a particular embodiment, the centrifuge can have the rotor shaft that drives the rotor. The rotor shaft can be connected in terms of drive technology to a motor at the end facing away from the rotor. The rotor shaft can be rotatably mounted in the front and rear walls. The rotor shaft can run through the rear wall and penetrate into an electronics space of the centrifuge in which the motor is arranged. The rear wall thus separates the rotor space of the centrifuge from the electronics space; in particular, the rear wall prevents ejected an liquid sample from penetrating the electronics space. This is advantageous because, in addition to the motor, further electrical or electronic devices, such as a control device, can be arranged in the electronics space, and the entry of a liquid sample into the electronics space should thus be prevented.

A central axis of the rotor shaft can run parallel to a base of the centrifuge. In particular, a central axis of the rotor shaft can run coaxially or parallel to the central axis of the rotor housing. The base can be formed by an underside of the lower shell or an underside of feet of the centrifuge. In addition, the central axis of the rotor shaft can run parallel to an insertion or removal direction of the sample carrier into the rotor. The removal direction can be directed opposite to the insertion direction. The insertion direction is understood to be the direction along which the sample carrier is moved in order to move the sample carrier from a centrifuge holder into the receiving section.

The front wall can have a passage through which the sample carrier can be introduced into the rotor, in particular a receiving section. The sample carrier can therefore be easily inserted into the rotor. In addition, the sample carrier can be removed from the rotor through the same passage. The centrifuge can have a displacement device by means of which the sample carrier is introduced into the rotor or removed from the rotor. As a result, in particular after the sample carrier has been introduced into a centrifuge holder, the sample carrier can be automatically introduced into the rotor by means of the displacement device. In addition, the sample carrier can be automatically removed from the rotor into the centrifuge holder using the displacement device.

The displacement device can be releasably connected to the sample carrier in order to displace the sample carrier. In particular, the displacement device can be magnetically connected to the sample carrier. To connect the sample carrier arranged in the centrifuge holder, the displacement device extends through the rotor, in particular the receiving section. The displacement device pulls the sample carrier into the receiving section of the rotor. In addition, the displacement device pushes the sample carrier back into the centrifuge holder after the washing operation has been completed. The displacement device is not arranged in the rotor space during the washing operation and the decontamination operation.

The centrifuge may have a flap for closing the passage of the front wall. The passage can be closed, in particular completely, with the flap when the centrifuge is in washing mode. In addition, the flap can close the passage, in particular completely, when the centrifuge is in decontamination mode. In decontamination mode, the radiation source emits radiation to kill the biological particles, in particular microorganisms, that have remained in the rotor space.

In a particular embodiment, the rotor housing can accommodate the at least one radiation source and/or be connected, in particular firmly, to the at least one radiation source. A fixed connection is a connection in which the two components connected to one another cannot move relative to one another. For this purpose, the at least one radiation source can be arranged on or in the rotor housing. In particular, the rotor housing can have a recess in which the at least one radiation source is arranged and/or into which the radiation source extends at least partially. It is advantageous if the front wall and/or the rear wall have a recess in which at least one radiation source is arranged and/or into which the radiation source extends at least partially. The shell, in particular the upper and/or lower shell, can have a recess in which at least one radiation source is at least partially arranged.

The centrifuge can have a cover that fluidly separates the radiation source from the rotor space. This prevents the radiation source from coming into contact with the ejected liquid sample. The cover can be configured such that it transmits radiation from the radiation source. The cover can be glass, preferably a glass with high transmission in the wavelength range of the radiation source, such as quartz glass. The cover may be positioned flush with the inner surface of the rotor housing so that it does not protrude from the rotor housing.

The centrifuge can have at least one electrical lead for electrically connecting the radiation source to an energy source, wherein the electrical lead runs at least partly through the front wall and/or rear wall. For this purpose, the front and/or rear wall has corresponding bore holes. The electrical lead can also run through the lower and/or upper shell. The energy source can be arranged in the electronics space of the centrifuge.

In a particular embodiment, the radiation source can be aligned such that the radiation hits the rotor and/or the inner surface of the rotor housing, in particular directly. This offers the advantage that rotor areas and/or inner surface areas of the rotor housing where it is known that the liquid sample is deposited can be irradiated in a targeted manner.

In an embodiment in which the centrifuge has multiple radiation sources, at least one first radiation source or multiple first radiation sources can be arranged on or in the front wall and at least one second radiation source or multiple second radiation sources can be arranged on or in the rear wall. Two to four first radiation sources can thus be provided on or in the front wall and two to four second radiation sources on or in the rear wall. The number of radiation sources attached to the front wall may be different from the number of radiation sources attached to the rear wall.

The radiation sources can be arranged such that they are not arranged mirror-symmetrically to one another in relation to the normal plane to the rotor shaft. This ensures in a simple manner that all areas of the rotor and/or the inner surface of the rotor housing can be exposed to radiation.

The centrifuge can have at least two radiation sources. It has been recognized that at least two, in particular exactly two, radiation sources are advantageous because when a single radiation source is used, there are many shaded areas in the rotor housing. Providing at least one further radiation source can reduce the shaded areas. The radiation sources can be arranged mirror-symmetrically to one another with respect to a mirror plane that contains a central axis of the rotor shaft. The mirror plane can extend along a gravitational direction. Such an arrangement offers the advantage that essentially all or the entire interior of the rotor housing can be exposed to radiation in a short time. This offers the advantage of ensuring that ejected biological particles can be quickly killed and/or inactivated. A short decontamination operation is desirable so that the centrifuge can soon be used for a washing operation. In decontamination mode, the biological particles, such as microorganisms, are irradiated with a minimum dose of radiation in order to kill or inactivate a defined minimum proportion of the population. The dose can be increased over the duration of the irradiation. In order to reduce the minimum exposure time, it is advantageous to avoid shaded areas and areas with weak intensity. Thus, the decontamination operation can be significantly shortened.

The at least two radiation sources can be connected to the shell, in particular the upper shell. In particular, the at least two radiation sources can be arranged on or in the shell, in particular the upper shell. The shell, in particular the upper and/or the lower shell, can have at least two openings. Each of the openings serves to accommodate at least part of a radiation source and/or enables the rotor space to be irradiated by the radiation source. The radiation sources can be arranged such that the emitted radiation runs in the direction of the central axis of the rotor housing. In particular, the radiation sources can be aligned in such a way that the emitted radiation or a large part of the emitted radiation first hits the rotor and/or the rotor shaft. The radiation source has an opening angle so that the rotor is irradiated completely or substantially completely in the axial direction and/or radial direction with respect to the central axis. Since the rotor has openings, some of the radiation can pass through the rotor and irradiate the inner housing surface behind it. Since the rotor and the inner housing surface are made of a highly reflective material and the surface scatters strongly, the direct radiation is reflected and/or scattered by the above-mentioned surfaces. By rotation of the rotor, the radiation can be distributed within the rotor space.

The radiation sources can be arranged in a region of the shell, wherein the region of the shell is arranged such that a plane which is perpendicular to the direction of gravity and includes a part of the region of the shell contains the central axis of the rotor shaft or is arranged offset from the central axis of the rotor shaft. The plane can be arranged offset from the central axis in the direction of gravity. A distance between the plane and the central axis in the direction of gravity can be in a range between 0 and 50%, in particular between 1 and 25%, preferably 1 and 15%, of the radial distance between the central axis and the rotor housing. Such positioning of the radiation sources has the advantage that it prevents the radiation sources from being arranged in an area of the rotor housing in which the ejected liquid sample is located. In particular, such placement of the radiation sources prevents liquid samples from reaching the cover and deposits from thus forming on the cover, which can impair the efficiency of the decontamination operation. The arrangement of the radiation sources described above also offers the advantage that the cover is almost vertical in the area mentioned, which further reduces the risk of deposits.

When two radiation sources are used, they can be arranged offset from one another in a tangential direction in a range between 160° to 200°, in particular 180°. Thus, better irradiation of the rotor space is achieved if the radiation sources are arranged as far apart as possible. On the other hand, when three radiation sources are used, the radiation sources can be offset from one another in a range between 100 to 140°, in particular 120°.

Radiation emitted by at least one first radiation source can have a different wavelength than radiation emitted by at least one second radiation source. This achieves the result that different biological particles are killed or inactivated particularly efficiently. This results from the fact that a wavelength at which a biological particle can be killed or inactivated particularly efficiently may depend on the type of biological particle.

As described above, the rotor has a receiving section into which the sample carrier that does not belong to the centrifuge can be inserted. In the inserted state of the sample carrier, the rotor can be connected to the sample carrier in a rotationally fixed manner. For this purpose, the receiving section can have a base wall, in particular a rectangular one, and two rails, in particular U-shaped rails.

The rotor can also have a further receiving section for receiving a further sample carrier. The further receiving section can lie diametrically opposite the receiving section in relation to the rotor shaft. The rotor can therefore accommodate multiple sample carriers. To receive the sample carrier, the rotor is rotated into a receiving position in which the sample carrier can be inserted into the respective receiving section of the rotor. The further receiving section can be designed to be identical to the receiving section.

The centrifuge can have a reflection body. According to one embodiment, the reflection body can be introduced into the receiving section of the rotor during a decontamination operation. The reflection body can be arranged in the receiving section instead of the sample carrier. The reflection body can have at least one surface for reflecting radiation. The surface can have a high reflectance in the wavelength range of the radiation source. In particular, the surface can have a higher reflectance than the rotor and/or the inner housing surface. The reflection body can be removed from the rotor after the decontamination process.

The surface of the reflection body and/or the rotor can have a reflection surface that is aligned such that it reflects radiation emitted by the radiation source into a predetermined rotor housing area. Likewise, at least part of the inner housing surface can be aligned such that it reflects the radiation emitted by the radiation source into a predetermined rotor housing area and/or a predetermined rotor area. The inner housing surface, in particular the previously mentioned part of the inner housing surface, can be coated with a material which has a high reflectance in the wavelength range of the radiation source. In particular, the material can have a higher reflectance than the uncoated rotor housing area. In addition, the type of coating can also influence the radiation direction in which reflected radiation is directed.

The predetermined rotor housing area and/or rotor area is understood to be an area of the rotor housing or a rotor area which would not be exposed to radiation during a decontamination operation without the provision of the reflection body and/or the reflection surface of the rotor and/or the part of the inner housing surface. This means that areas of the rotor and/or rotor housing that were previously inaccessible to radiation can be exposed to radiation.

In another embodiment, a reflection body can be connected to the rotor in a rotationally fixed manner. In this embodiment, the reflection body is not attached to the receiving section of the rotor, but is connected to the rotor in a rotationally fixed manner, regardless of whether the centrifuge is operated in washing mode or decontamination mode. The reflection body can be attached to a rotor side which is arranged offset in a tangential direction in relation to the receiving section, in particular to a base wall. In particular, the rotor side can be arranged offset by 90° in relation to the receiving section. This means that an angle between a plane containing the rotor side and another plane containing the receiving section is 90°.

The reflection body can have a reflection surface. The reflection surface can extend in the axial direction of the central axis of the rotor shaft. In addition, the reflection surface can run transversely to the central axis of the rotor shaft. This means that a radial distance of one end of the reflection surface from the central axis is smaller than a radial distance of another end of the reflection surface from the central axis. Providing an inclined reflection surface offers the advantage that the inclination can be selected such that areas of the rotor housing are provided with radiation that would otherwise only receive low levels of radiation. A “low level of radiation” is understood to mean that the radiation intensity of the radiation supplied to the respective area is lower than the average radiation intensity and/or is lower than radiation which is supplied to the remaining area of the rotor space. Providing the reflection body achieves the result that the minimum radiation intensity is increased, i.e. more radiation reaches the areas that are difficult to access. This shortens the duration of a cleaning step.

The centrifuge can have at least two reflection bodies. The reflection bodies can be arranged on opposite sides of the rotor. The two reflection bodies can each have a reflection surface. The reflection surfaces can have the same inclination. Alternatively, the reflection surfaces can differ from one another in their inclination and/or inclination direction. In particular, one reflection body can be connected in a rotationally fixed manner to one rotor side and another reflection body can be connected in a rotationally fixed manner to another rotor side. The two rotor sides can lie opposite each other with respect to the central axis of the rotor shaft. Such an embodiment reflects the emitted radiation particularly well.

In addition to the reflection body which is connected to the rotor in a rotationally fixed manner, a reflection body can be introduced into the receiving section. Such an embodiment reflects the radiation into the interior of the rotor housing particularly well.

In a particular embodiment, the at least one radiation source can be connected to the rotor in a rotationally fixed manner. Thus, the radiation source rotates together with the rotor. This offers the advantage that a large area of the rotor housing can be irradiated. The radiation source arranged on or in the rotor can be aligned such that the radiation is emitted radially outwards from the rotor. Outwards means that the radiation is directed from the rotor to the rotor housing.

The receiving section and/or the further receiving section can accommodate at least one radiation source. In particular, at least one radiation source can be arranged on or in the receiving section. In addition, at least one radiation source can be arranged on or in the further receiving section. An embodiment is advantageous in which the at least one radiation source is arranged on or in the base wall and/or on or in at least one of the two rails. The radiation source can be arranged in a recess of the base wall and/or in a recess of the rail. A cover can be present which fluidly separates the radiation source from the rotor space so that the radiation source does not come into contact with the ejected liquid sample.

The centrifuge may have an electrical lead for connecting the radiation source to an energy source. The electrical lead can run at least partly through the rotor shaft. A loop ring can be used to electrically connect the electrical energy source to the rotating rotor shaft.

In a particular embodiment, the centrifuge can have multiple radiation sources. The radiation sources can be arranged at a distance from one another in the axial direction, in particular along the receiving section and/or further receiving section. In addition, radiation sources can be arranged at a distance from one another in a tangential direction, in particular along the receiving section and/or the further receiving section. The multiple radiation sources can extend parallel to a central axis of the rotor shaft.

The radiation source can be a punctiform radiation source. Alternatively, the radiation source can be designed to be linear. In addition, multiple punctiform radiation sources can be arranged along a line, in particular a straight or curved line. As a result, the radiation source or sources can be arranged or designed such that the largest possible area, in particular the entire area, of the rotor and/or the rotor housing is exposed to radiation.

The radiation source can be a UV-C radiation source. A UV-C radiation source offers the advantage that it kills and/or decomposes biological particles particularly well. A UV-C radiation source can emit radiation in the wavelength range between 200 nm and 280 nm.

The inner surface of the rotor housing and/or a rotor surface and/or a rotor shaft surface can be coated. In particular, the surfaces of the rotor housing, the [omission], and/or the rotor shaft that can come into contact with liquid sample can be coated. It is advantageous if the aforementioned surfaces are anodized. An anodized surface has the advantage that it scatters the emitted radiation more strongly than a non-anodized surface, particularly an aluminum surface. A further advantage is that an anodized surface is more chemically resistant than uncoated or non-anodized materials, such as aluminum.

In a particular embodiment, the radiation source can have an emitter for emitting radiation. The emitter can be an LED emitter. In addition, the emitter can be connected to the rotor housing in a heat-conducting manner by means of a heat transfer section. The heat-conducting connection offers the advantage that the heat that occurs can be conducted away from the emitter to the rotor housing. A heat transfer section is understood to be a section that consists of solid and/or liquid components and/or has no gas components. This ensures good heat conduction from the emitter to the rotor housing.

The emitter can be attached to a metal conducting element of the heat transfer section. The heat transfer section can have a conductive paste, by means of which the metallic conducting element is connected to the rotor housing in a heat-conducting manner. This means that providing the heat transfer section enables heat conduction from the emitter to the rotor housing to occur through non-gaseous components, thereby improving heat dissipation.

The radiation source can also have a printed circuit board. A printed circuit board is often referred to as a PCB and is used to carry electronic components. The metallic conducting element can be connected directly to the printed circuit board. “Direct” means that no other components are arranged between the metal conducting element and the printed circuit board. The metallic conducting element can be a coating, in particular a copper layer, attached to the printed circuit board and can thus be connected to the printed circuit board in a materially bonded manner. Alternatively, the metallic conducting element and the printed circuit board can be designed as separate components. In this case, the two components can be mechanically connected to one another. The printed circuit board can be electrically connected to the emitter.

In a particular embodiment, the control device of the centrifuge can ensure that the radiation source does not emit any radiation during a washing operation. Alternatively or additionally, the control device can cause the radiation source to emit radiation when the rotor does not carry a sample carrier. In this case, radiation is only emitted if no sample carrier is arranged in the receiving section of the rotor. The previously described control has the advantage that it ensures that no biological particles remaining in the sample carrier are killed.

The control device can cause the radiation to be emitted before or after a washing operation. Alternatively or additionally, the radiation can be emitted at predetermined times and/or for a predetermined period of time. This allows the decontamination operation to be carried out overnight to obtain a decontaminated centrifuge at the start of each working day. In addition, the control device can cause the radiation source to be switched off as soon as the flap does not cover the passage, in particular completely.

In addition, the control device can cause the rotor to be brought, in particular rotated, into a predetermined position in the decontamination operation, which differs from a position in which the sample carrier can be inserted into the rotor or ejected from the rotor. This offers the advantage that the rotor can be rotated into a position in which the rotor provides little shade to the rotor housing. In this embodiment, the rotor is not rotated in decontamination mode.

The control device may also cause the rotor to rotate during the decontamination operation. The control device can cause the rotor to rotate for the duration of the entire decontamination operation. The rotor rotation ensures that radiation reaches areas of the rotor housing that were originally shaded by the rotor. The rotor rotation thus ensures that more radiation is supplied to the areas of the rotor housing that are shaded, for example by the rotor. The rotor is used as a scatterer/reflector that scatters/reflects the radiation into the rotation space. In washing mode, a rotor speed can be higher than in a decontamination operation in which radiation is emitted. Rotating the rotor at low speed has the advantage that the radiation can be reflected by the rotor into a large part of the rotor housing.

The subject matter of the invention is shown schematically in the figures, wherein elements that are the same or have the same effect are usually provided with the same reference symbols. In the figures:

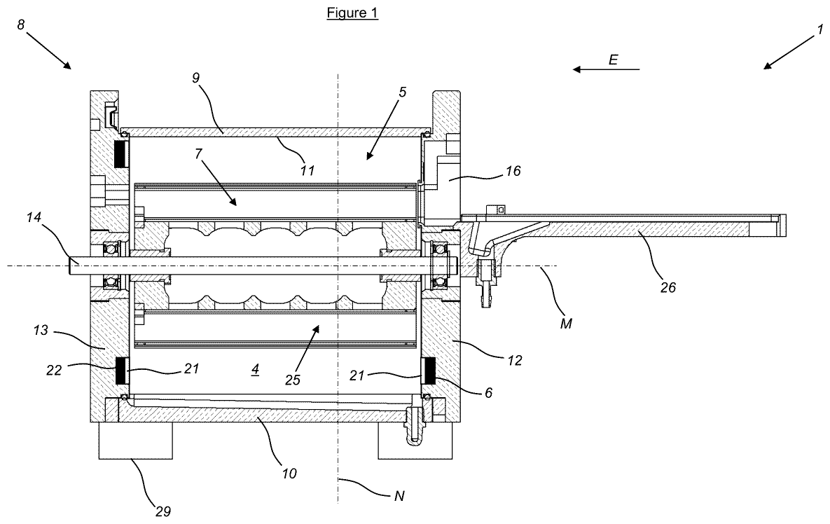

FIG. 1 is a sectional view of part of a centrifuge according to the invention

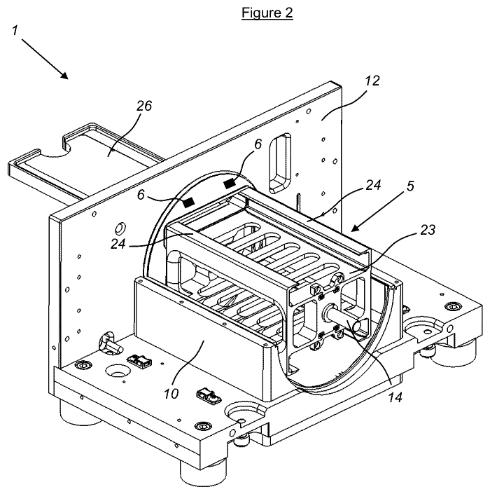

FIG. 2 is a perspective view of part of the centrifuge shown in FIG. 1 without the rear wall and upper shell,

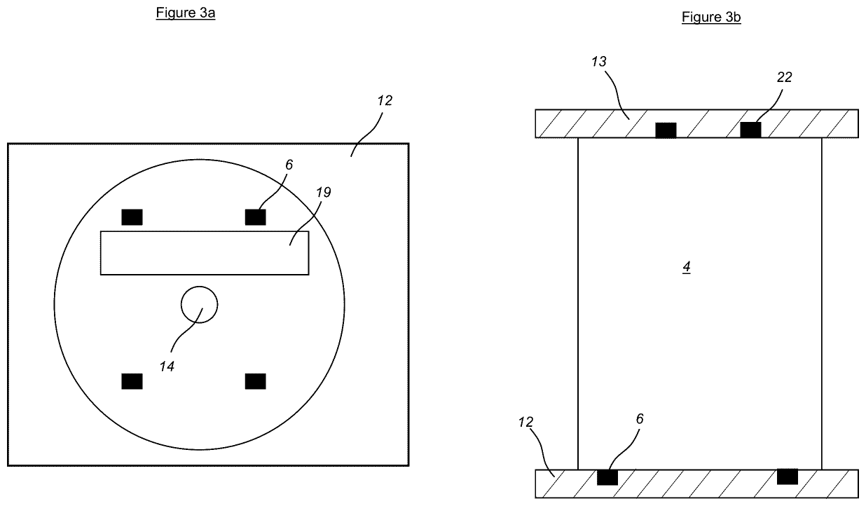

FIG. 3a is a plan view of the front wall,

FIG. 3b is a sectional view from above in a plane that has radiation sources arranged in the front and rear walls,

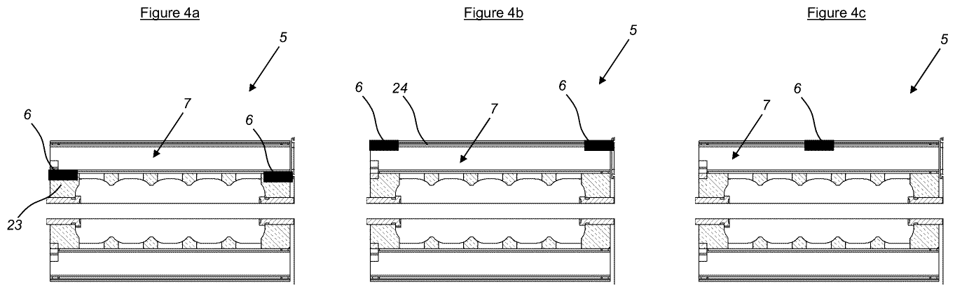

FIGS. 4a-f are sectional views of a rotor of the centrifuge in different embodiments,

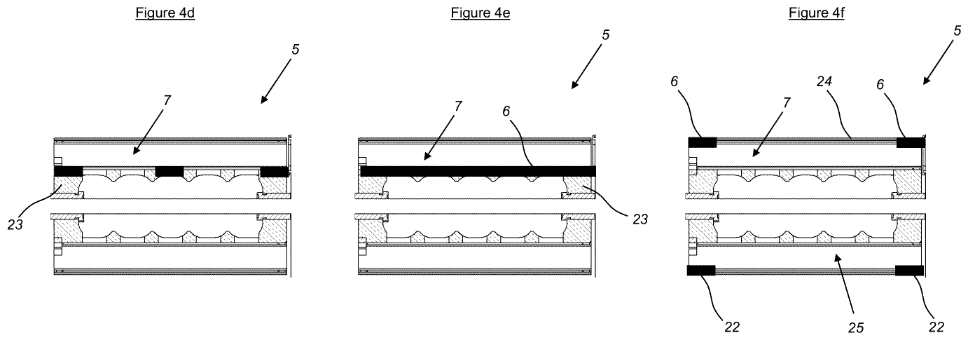

FIG. 5 is a perspective view of part of a centrifuge according to the invention with a different arrangement of the radiation sources,

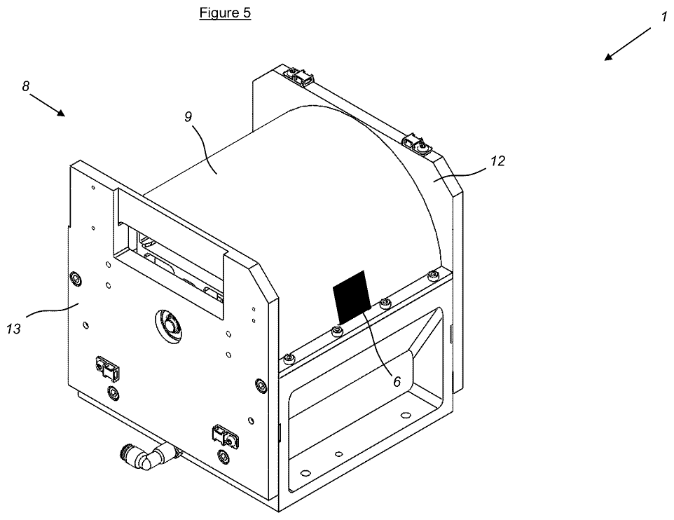

FIG. 6 is a sectional view of part of the centrifuge shown in FIG. 5 from the front,

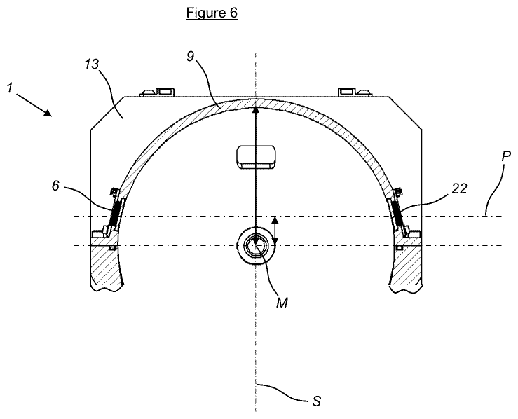

FIG. 7 is a perspective view of an upper shell of the centrifuge shown in FIG. 5

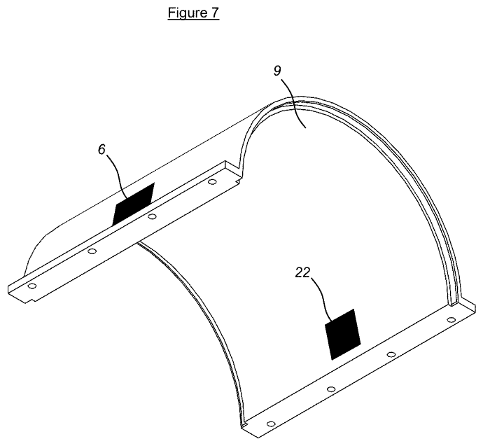

FIG. 8 is an enlarged view of a radiation source;

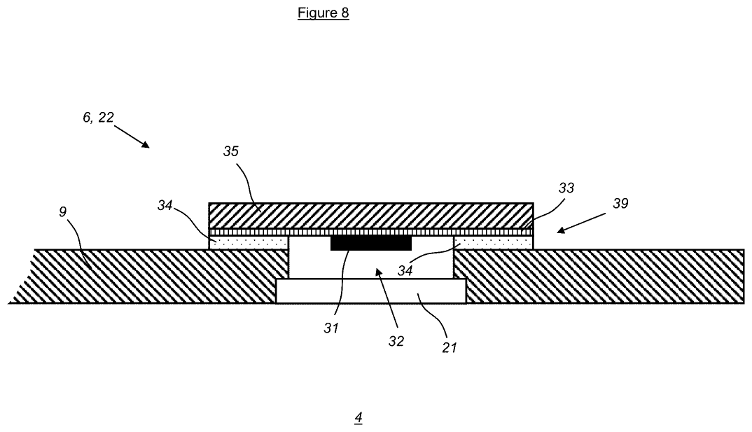

FIG. 9 is a perspective view of a rotor;

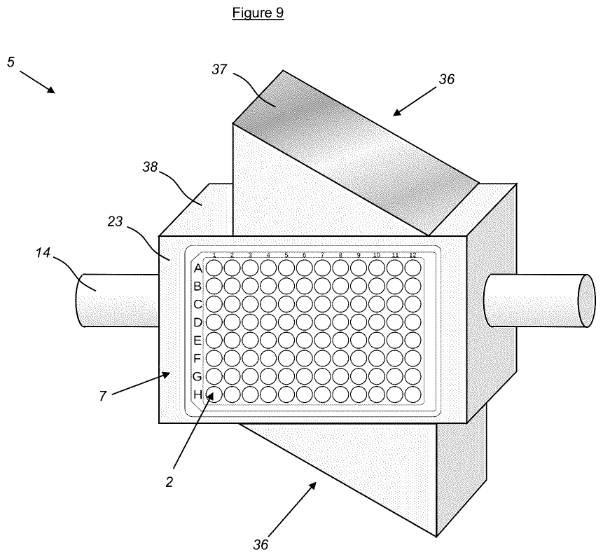

FIG. 10 is a perspective view of part of the centrifuge with electronics space,

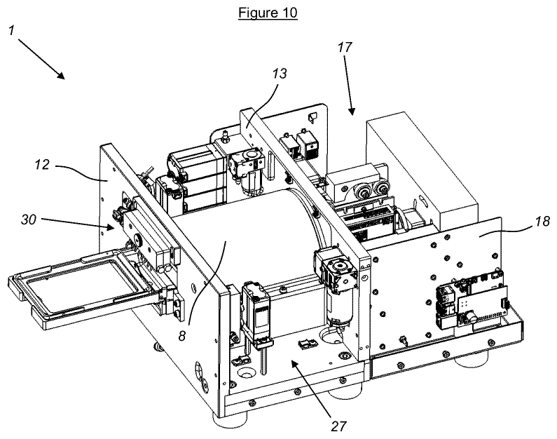

FIG. 11 is a perspective view of the centrifuge.

A centrifuge 1 shown in FIG. 1 is used to rotate a sample carrier 2 shown in FIG. 11. The centrifuge 1 has a rotor 5, which has a receiving section 7 for receiving the sample carrier 2. In addition, the centrifuge 1 has a rotor space 4, in which the rotor 5 is arranged, and multiple radiation sources, namely a first radiation source 6 and a second radiation source 22, for emitting radiation into the rotor space 4.

The radiation sources 6, 22 are designed such that they emit radiation with a wavelength of a maximum of 350 nm (nanometers). In particular, the radiation sources 6, 22 can be designed such that they emit radiation with a wavelength in the range between 100 nm, in particular 200 nm, to 350 nm, in particular 315, preferably 280. Radiation sources 6, 22 which emit radiation with a wavelength of 254 nm or in a range between 260 nm and 265 nm are quite particularly advantageous. The first radiation sources 6 and the second radiation sources 22 can emit radiation with different wavelengths. The radiation sources 6, 22 can each be a UV-C radiation source.

The first radiation sources 6 can be arranged in the front wall 12. In particular, the front wall 12 can have recesses, in each of which a first radiation source 6 is arranged. The centrifuge 1 can have a cover 21 that separates the first radiation source 6 from the rotor space 4, in particular fluidly. The cover 21 is also arranged in the recess. The second radiation sources 22 can be arranged in the rear wall 13. In particular, the rear wall 13 can have recesses in which a second radiation source 22 is arranged. The centrifuge 1 can have a cover 21 which separates the second radiation source 22 from the rotor space 4, in particular fluidly.

The rotor space 4 is delimited by a rotor housing 8 of the centrifuge 1. The rotor housing 8 has an upper shell 9 and a lower shell 10. Both shells 9, 10 are detachably connected to one another. In addition, the rotor housing 8 has the front wall 12 and the rear wall 13. Only part of the front wall 12 and part of the rear wall 13 delimit the rotor space 4.

The radiation sources 6, 22 are aligned such that they emit radiation into the rotor space 4. The radiation sources 6, 22 are aligned such that the emitted radiation is reflected on the rotor 5 and/or an inner housing surface 11. The inner housing surface 11 is formed by the surfaces of the upper shell 9, the lower shell 10, the front wall 12, and the rear wall 13 facing the rotor space 4. The inner housing surface 11 is cylindrical in a normal plane N, which is perpendicular to a central axis M and includes a part of the upper shell 9 and the lower shell 10.

The centrifuge 1 has a centrifuge holder 26 on which a user of the centrifuge 1 places the sample carrier 2. A displacement device, not shown in the figures, then moves the sample carrier 2 into the receiving section 7 of the rotor 5 along an insertion direction E or moves the sample carrier 2 from the receiving section 7 into the centrifuge holder 26 along a removal direction that is opposite to the insertion direction E. The centrifuge holder 26 projects from the front wall 12, in particular in the axial direction. The displacement device is driven by a displacement motor, not shown, which is arranged in the electronics space shown in FIG. 5. The displacement device may have a wound belt that is driven by the displacement motor. For coupling with the sample carrier 2 arranged in the centrifuge holder 26, the displacement device moves through the rotor space 4 and is connected to the sample carrier 2, in particular magnetically.

The front wall 12 has a passage 16 through which the sample carrier 2 can be introduced into the receiving section 7 of the rotor 5. The sample carrier 2 is removed from the receiving section 7 through the passage 16. The passage 16 is arranged radially offset with respect to a central axis M of the rotor housing 8. In particular, the passage 16 is designed such that it is arranged completely offset from the central axis M, i.e. the central axis M does not run through the passage 16.

The rotor 5 is connected to a rotor shaft 14 in a rotationally fixed manner. The rotor shaft 14 is rotatably mounted in the front wall 12 and the rear wall 13 and extends through the rear wall 13. The rotor shaft 14 is connected in terms of drive technology to a motor not shown in the figures. The motor is arranged in an electronics space 17 shown in FIG. 5.

In the position shown in FIG. 1, the rotor 5 is rotated such that a sample carrier 2 can be introduced into the receiving section 7. The rotor 5 has a further receiving section 25, which is diametrically opposite the receiving section with respect to the central axis M. The further receiving section 25 is designed to be identical to the receiving section 7. In this respect, the rotor 5 can accommodate two sample carriers 2.

FIG. 2 shows a perspective view of a part of the centrifuge 1 shown in FIG. 1 without the rear wall and upper shell. The lower shell 10 has a semi-cylindrical inner housing surface 11 in the normal plane N. After the upper shell 9 has been mounted, the inner housing surface 11 is cylindrical in the normal plane N.

The receiving section 7 has a base wall 23 and two U-shaped rails 24. After the sample carrier 2 has been introduced into the receiving section 7, the sample carrier 2 rests on the base wall 23. The U-shaped rails 24 prevent the sample carrier 2 from moving in the radial direction.

FIG. 3a shows a plan view of the front wall 12. The front wall 12 has four first radiation sources 6. The first radiation sources 6 are arranged spaced apart from one another in the tangential direction with respect to the central axis M. In addition, the front wall 12 has the passage 16, which can be closed by means of a flap 19.

FIG. 3b shows a sectional view from above in a plane, in particular a horizontal plane, which has radiation sources 6, 22 arranged in the front wall 12 and rear wall 13. The rotor 5 is not shown in FIG. 3b; only the rotor space 4 can be seen. Although this is not apparent from FIG. 3b, the rear wall 13 has four second radiation sources 22. The second radiation sources 22 are arranged spaced apart from one another in a tangential direction with respect to the central axis M. The first and second radiation sources 6, 22 are arranged such that they are not arranged mirror-symmetrically to one another with respect to a normal plane N shown in FIG. 1. This means that the radiation sources are arranged offset from one another, so that a large part of the inner surface 11 of the rotor housing 8 can be irradiated. In alternative embodiments, not shown, the radiation sources can be arranged mirror-symmetrically to one another with respect to the normal plane N.

FIGS. 4a-f show sectional views of a rotor 5 of the centrifuge 1 in different embodiments. In the embodiment of the rotor 5 shown in FIG. 4a, two first radiation sources 6 are arranged in the base wall 23 of the rotor 5.

The radiation sources 6 are each arranged at one end of the rotor 5. In addition, the radiation sources 6 are arranged at a distance from one another in the axial direction.

In the embodiment of the rotor 5 shown in FIG. 4b, two first radiation sources 6 are arranged in at least one of the two rails 24. The radiation sources 6 are each arranged at one end of the rotor 5. In addition, the radiation sources 6 are arranged at a distance from one another in the axial direction.

In the embodiment of the rotor 5 shown in FIG. 4c, a first radiation source 6 is arranged in at least one of the two rails 24. The first radiation source 6 has a larger radiation angle than the radiation sources in the embodiments shown in FIGS. 4a and 4b.

The embodiment of the rotor 5 shown in FIG. 4d differs from the embodiment shown in FIG. 4a in that three first radiation sources 6 are present. The third radiation source 6 is also arranged in the base wall 23.

The embodiment of the rotor 5 shown in FIG. 4e has a linear radiation source 6 which extends in the axial direction along the entire length of the base wall 23.

The embodiment of the rotor 5 shown in FIG. 4f differs from the embodiment shown in FIG. 4b in that additional radiation sources 22 are present. The second radiation sources 22 are arranged in the further receiving section 25. In particular, the second radiation sources 22 are arranged in at least one of the rails 24 of the further receiving section 25. The second radiation sources 22 are each arranged at one end of the rotor 5. In addition, the second radiation sources 22 are arranged at a distance from one another in the axial direction.

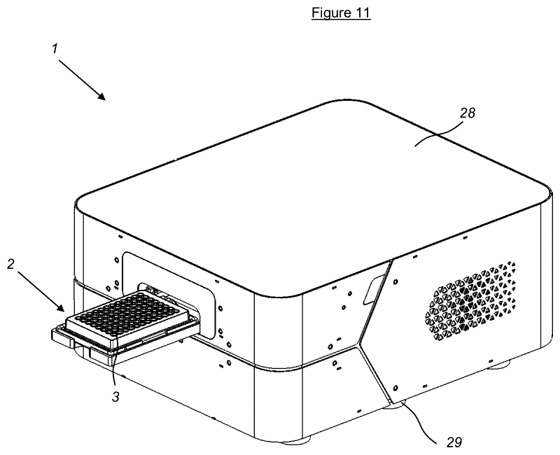

The rotors 5 shown in FIGS. 4a to 4f can be used in the centrifuge 1 shown in FIGS. 1, 2, 5, 11, and 12.

FIG. 5 shows a perspective view of a part of a further centrifuge 1 according to the invention. The centrifuge 1 differs from the centrifuges 1 described above in the arrangement of the radiation sources 6, 22. In the embodiment shown in FIG. 5, a first radiation source 6 is attached to the upper shell 9. A second radiation source 22, not shown, is also attached to the upper shell 9. The structure of the first and second radiation sources 6, 22 is shown in FIG. 8. The first and second radiation sources 6, 22 are identical.

FIG. 6 shows a sectional view of the part of the centrifuge 1 shown in FIG. 5 from the front and FIG. 7 shows a perspective view of the upper shell 9 of the further centrifuge 1 shown in FIG. 5. The centrifuge 1 has two radiation sources 6, 22. Both radiation sources 6, 22 are attached to the upper shell 9. As can be better seen in FIG. 8, the two radiation sources 6, 22 are arranged on the upper shell 9 and/or are partly arranged in an opening 32 of the upper shell 9. The two radiation sources are arranged mirror-symmetrically to one another with respect to a mirror plane S. The mirror plane S has the central axis M of the rotor housing 8 and also extends in the direction of gravity. The two radiation sources 6, 22 are aligned such that the emitted radiation extends in the direction of the central axis M of the rotor housing 8. In other words, the emitted radiation hits the rotor 5, not shown, before it is reflected by an inner housing surface 11.

Furthermore, it can be seen from FIG. 6 that the two radiation sources 6, 22 are arranged in an area of the upper shell 6 such that a plane P exists, whose radial distance from the central axis M, i.e. in the direction of gravity, is smaller than 50% of the radial distance between the central axis M and the rotor housing 8. The radial distance between the central axis M and the rotor housing 8 is understood to be the distance in the direction of gravity. The plane P runs perpendicular to the direction of gravity and contains part of the two radiation sources 6, 22. In FIG. 6, both the radial distance between the central axis M and the plane P and the radial distance between the central axis M and the rotor housing 8 are symbolized as a double arrow. The two radiation sources 6, 22 are arranged offset from one another in a tangential direction in a range between 160° to 200°, preferably 180°.

FIG. 8 shows an enlarged view of a radiation source 6, 22. The radiation source 6, 22 has an emitter 31, such as an LED, by means of which radiation is emitted into the rotor space 4. The radiation source 6, 22 is arranged on the upper shell 9, with the emitter 31 partially extending into an opening 32 of the upper shell 9.

The radiation source 6, 22 has a heat transfer section 39, by means of which the emitter 31 is connected to the upper shell 9 in a heat-conducting manner. The heat transfer section 39 has a metallic conducting element 33, wherein the emitter 31 is arranged on the metallic conducting element 33. In addition, the radiation source 6, 22 has a printed circuit board 35. The metallic conducting element 33 is connected to the printed circuit board 35 in a materially bonded manner. In addition, the metallic conducting element 33 is connected directly to the upper shell 9 by means of conductive pastes 34. As a result, heat conduction from the emitter 31 to the upper shell 9 only occurs via non-gaseous sections of the heat transfer section 39.

FIG. 9 shows a perspective view of a rotor 5. The rotor 5 differs from the rotor that is used in the embodiments shown in FIGS. 1 to 8 in that the rotor 5 has two reflection bodies 36. Both reflection bodies 36 are each arranged on a rotor side 38. The rotor side 38 is offset in a tangential direction from the receiving section 7 for receiving the sample carrier 2. In particular, the rotor side 38 runs perpendicular or substantially perpendicular to the base wall 23 of the rotor 5.

The two reflection bodies 36 are arranged on opposite rotor sides 38. The two rotor sides 38 lie opposite each other, in particular radially, with respect to the central axis of the rotor shaft 14. Both reflection bodies 36 each have a reflection surface 37, such as a mirror, by means of which the radiation is reflected. The reflection surface 37 has a higher reflectance than the other components of the rotor 5 and/or rotor shaft 14. The reflection surface 37 runs transversely to one [omission] of the central axis of the rotor shaft 14.

FIG. 10 shows a perspective view of a part of the centrifuge 1 with the electronics space 17. The electronics space 17 is separated from the rotor space 4 and a front space 27 of the centrifuge 1 by the rear wall 13. A control device 18 for controlling a washing operation and/or a decontamination operation is arranged in the electronics space 17. In addition, the displacement device for introducing or removing the sample carrier 2 into the rotor 5 or out of the rotor 5 and the displacement motor are arranged in the electronics space 17. The rotor housing 8 is arranged in the front space 27. In addition, the centrifuge 1 can have a dispensing device 30, which is arranged on the front wall 12.

The rotor housing 8, the rotor 5, and the radiation sources 6, 22 can be designed or arranged similarly to the embodiments described in FIGS. 1-9.

FIG. 11 shows a perspective view of the centrifuge 1. The centrifuge 1 has a centrifuge housing 28 which encloses the front space 27 and the electronics space 17. The rotor housing 8 is also arranged in a space enclosed by the centrifuge housing 28. In addition, the centrifuge 1 has feet 29 for standing the centrifuge 1 on a floor or work table or the like. The sample carrier 2 is designed as a microtiter plate, which is arranged in the centrifuge holder 26. The microtiter plate has a plurality of containers 3.

The centrifuge 1 can be designed similarly to one of the embodiments described in FIGS. 1 to 10.

### LIST OF REFERENCE SYMBOLS

1 Centrifuge
2 Sample carrier
3 Container
4 Rotor space
5 Rotor
6 First radiation source
7 Receiving section
8 Rotor housing
9 Upper shell
10 Lower shell
11 Inner housing surface
12 Front wall
13 Rear wall
14 Rotor shaft
16 Passage
17 Electronics space
18 Control device
19 Flap
21 Cover
22 Second radiation source
23 Base wall
24 Rail
25 Further receiving section
26 Centrifuge holder
27 Front space
28 Centrifuge housing
29 Feet
30 Dispensing device
31 Emitter
32 Opening
33 Metallic conducting element
34 Printed circuit board
35 Conductive paste
36 Reflection body
37 Reflection surface
38 Rotor side
39 Heat transfer section
E Insertion direction
M Central axis
N Normal plane
P Plane
S Mirror plane

1.-33. (canceled)

34. A centrifuge for rotating a sample carrier, which has at least one container for receiving a liquid sample, with a rotatable rotor, which has at least one receiving section for receiving the sample carrier, and a rotor space in which the rotor is arranged, the rotor space delimited by a rotor housing, wherein the centrifuge has at least one radiation source for emitting radiation into the rotor space with a wavelength of a maximum of 350 nm, and wherein the radiation source has an emitter which is connected in a heat-conducting manner to the rotor housing by means of a heat transfer section.

35. The centrifuge according to claim 34, wherein the rotor housing:
a. has an upper shell and a lower shell, which are releasably connected to one another, and which delimit the rotor space; and/or
b. has an upper shell which has a cylindrical inner housing surface; and/or
c. is configured such that it prevents the radiation from escaping from the rotor space.

36. The centrifuge according to claim 34, wherein the rotor housing has a front wall and a rear wall for delimiting the rotor space.

37. The centrifuge according to claim 36, wherein the centrifuge has a rotor shaft which drives the rotor, and:
a. is rotatably mounted in the front and rear walls; and/or wherein
b. a central axis of the rotor shaft runs into the rotor parallel to a base and/or parallel to an insertion direction of the sample carrier.

38. The centrifuge according to claim 36, wherein:
a. the front wall has a passage through which the sample carrier can be inserted into the rotor; and/or
b. the centrifuge has a flap for closing a passage in the front wall; and/or
c. the rear wall separates the rotor space from an electronics space of the centrifuge, in which a control device of the centrifuge is arranged.

39. The centrifuge according to claim 34, wherein:
a. the centrifuge has a displacement device for introducing the sample carrier into the rotor and/or for removing the sample carrier from the rotor; and/or
b. the centrifuge has a displacement device which is designed such that it extends through the rotor, for connecting the sample carrier arranged in a centrifuge holder; and/or
c. the centrifuge has a displacement device which is designed such that it is not arranged in the rotor space during a washing operation and a decontamination operation.

40. The centrifuge according to claim 34, wherein:
a. the rotor housing is connected to at least one radiation source; and/or
b. the at least one radiation source is arranged on or in the rotor housing; and/or
c. a front wall and/or a rear wall of the rotor housing has a recess in which at least one radiation source is arranged; and/or
d. an upper and/or lower shell of the rotor housing has a recess in which at least one radiation source is arranged.

41. The centrifuge according to claim 34, wherein the centrifuge has a cover which fluidically separates the radiation source from the rotor space and/or is configured such that it transmits radiation from the radiation source.

42. The centrifuge according to claim 40, wherein:
a. the centrifuge has at least one electrical lead for electrically connecting the radiation source to an energy source, wherein the electrical lead runs at least partly through the front wall and/or the rear wall; and/or
b. the radiation source is aligned such that the radiation hits the rotor and/or the inner housing surface.

43. The centrifuge according to claim 40, wherein multiple radiation sources are present, and wherein:
a. at least one first radiation source is arranged on or in the front wall and at least one second radiation source is arranged on or in the rear wall, and the radiation sources are arranged such that they are not mirror-symmetrical to one another in relation to a normal plane on a rotor shaft; and/or wherein
b. a radiation emitted by at least one first radiation source has a different wavelength than radiation emitted by at least one second radiation source.

44. The centrifuge according to claim 34, wherein:
a. at least two radiation sources are arranged mirror-symmetrically to one another with respect to a mirror plane, which contains a central axis of a rotor shaft; and/or
b. at least two radiation sources are connected to a shell of the rotor housing; and/or
c. the radiation sources are arranged in a region of a shell, wherein the region of the shell is arranged such that a plane which runs perpendicular to the direction of gravity and includes a part of the section of the shell contains the central axis of the rotor shaft or is arranged offset from the central axis of the rotor shaft.

45. The centrifuge according to claim 34, wherein:
a. the receiving section has a base wall and two rails; and/or
b. the rotor has a further receiving section for receiving a further sample carrier; and/or
c. the centrifuge has at least one reflection body which is introduced into the receiving section of the rotor, wherein the reflection body has at least one surface for reflecting radiation; and/or
d. the rotor has a reflecting surface which is aligned such that it reflects the radiation emitted by the radiation source into a predetermined housing area; and/or
e. a part of the inner housing surface is aligned such that it reflects the radiation emitted by the radiation source into a predetermined rotor housing area and/or a predetermined rotor area.

46. The centrifuge according to claim 34, wherein the centrifuge has at least one reflection body, wherein:
a. the reflection body is connected to the rotor in a rotationally fixed manner; and/or
b. the reflection body is attached to a rotor side which is arranged offset in a tangential direction in relation to the receiving section; and/or
c. the reflection body has a reflection surface which extends axially and/or runs transversely to a central axis of a rotor shaft; and/or
d. the centrifuge has at least two reflection bodies which are arranged on opposite rotor sides.

47. The centrifuge according to claim 34, wherein the at least one radiation source is connected to the rotor in a rotationally fixed manner.

48. The centrifuge according to claim 47, wherein:
a. the receiving section and/or a further receiving section receives at least one radiation source; and/or
b. the at least one radiation source is arranged on or in the receiving section; and/or
c. the at least one radiation source is arranged on or in the further receiving section.

49. The centrifuge according to claim 47, wherein:
a. the at least one radiation source is arranged on or in a base wall of the receiving section and/or on or in at least one of two rails of the receiving section; and/or
b. the radiation source is aligned such that the radiation from the rotor is emitted radially outwards; and/or
c. the centrifuge has an electrical lead for connecting the radiation source to an energy source, wherein the electrical lead runs at least partly through a rotor shaft; and/or
d. in that multiple radiation sources are present, which are arranged at a distance from one another in an axial direction, and/or which are arranged at a distance from one another in a tangential direction, and/or which extend parallel to a central axis of the rotor shaft.

50. The centrifuge according to claim 34, wherein:
a. the radiation source is a punctiform radiation source; and/or
b. the radiation source is a linear radiation source; and/or
c. multiple punctiform radiation sources are arranged along a line; and/or
d. the radiation source is a UV-C radiation source.

51. The centrifuge according to claim 34, wherein:
a. an inner housing surface of the rotor housing and/or a rotor surface and/or a rotor shaft surface is coated; and/or
b. the emitter is attached to a metallic conducting element of the heat transfer section; and/or
c. the heat transfer section has a conductive paste, by means of which a metallic conducting element of the heat transfer section is connected to the rotor housing in a heat-conducting manner; and/or
d. the radiation source has a printed circuit board which is connected directly to the metallic conducting element.

52. The centrifuge according to claim 34, wherein the centrifuge has a control device, which effects the following:
a. during a washing operation, the radiation source does not emit any radiation; and/or
b. the radiation source emits radiation when no sample carrier is arranged in the rotor; and/or
c. the rotor is brought into a predetermined position in a decontamination operation, which differs from a position in which the sample carrier can be inserted into the rotor or ejected from the rotor; and/or
d. the rotor is rotated during the decontamination operation.
